(19) United States
(12) Patent Application Publication
(10) Pub. No.: US 2021/0027884 A1
(43) Pub. Date: Jan. 28, 2021

(54) SYSTEMS AND METHODS FOR ROUTING RADIOLOGY EXAMS

(71) Applicant: Intelerad Medical Systems Incorporated, Seattle, WA (US)

(72) Inventor: Chris Wood, Seattle, WA (US)

(21) Appl. No.: 16/930,804

(22) Filed: Jul. 16, 2020

Related U.S. Application Data
(60) Provisional application No. 62/877,046, filed on Jul. 22, 2019.

(51) Int. Cl. G16H 40/20; G16H 50/20; G16H 70/20; G06Q 10/06; G16H 30/40; A61B 6/00; A61B 5/055; A61B 8/08; G06T 7/00

(52) U.S. Cl. CPC G16H 40/20; G16H 50/20; G16H 70/20; G06Q 10/06311; G16H 30/40; G06T 2207/30004; A61B 6/5217; A61B 5/055; A61B 8/5223; G06T 7/0012; G06Q 10/06312

(57) ABSTRACT

A system and method to review a radiology exam is described herein. The system is programmed to analyze the radiology exam by image processing or artificial intelligence and then, based on the analysis, re-order a review queue, notify a reviewer to review the radiology exam, assign the radiology exam to a specific reviewer, the like, or combinations or multiples thereof. The analysis and subsequent re-order, notification, or assignment can be controlled by review settings. The review settings can also turn the image processing or artificial intelligence on or off.

### CROSS-REFERENCE TO RELATED APPLICATIONS

This application claims the benefit of pending U.S. Provisional Patent Application Ser. No. 62/877,046, filed Jul. 22, 2019, the contents of which are herein incorporated by reference in their entirety.

### BACKGROUND

As of 2015, a radiologist may need to review one radiology image every three to four seconds to meet workload demands for 255 uninterrupted eight-hour work days per year. Therefore, patient and physicians can wait hours, or even days, for radiology reports. This back up can cause delays in the review, analysis, and evaluation of radiology exams.

Furthermore, a general radiologist or radiology sub-specialist may have left the office for the day and will not review any radiology exams until the following day at the earliest. This can cause a delay or a further delay in the review, analysis, and evaluation of the radiology exams.

In some cases, these delays can have a significant impact on medical treatment or patient prognosis. For example, delayed detection of a pneumothorax (i.e., a collapsed lung) can result in a life threatening condition or chronic respiratory or cardiac events.

In some cases, these delays can increase patient anxiety. For example, a patient who fall and hit his or her head may be concerned about an intracranial hemorrhage and want medical assurance of the condition and prognosis as soon as possible.

What is needed is a system to effectively and efficiently review radiology exams.

### BRIEF DESCRIPTION OF THE DRAWINGS

FIG. 1 illustrates an example system for reviewing a radiology exam.

FIG. 2 illustrates an example radiology server.

FIG. 3 illustrates an example method for reviewing a radiology exam.

FIG. 4 illustrates an example computing device or system.

### DETAILED DESCRIPTION

Exam processing algorithms can review, analyse, and evaluate radiology exams. For example, an algorithm can identify a possible pneumothorax within a chest x-ray or an intracranial hemorrhage within a CT scan. In response, the system will then communicate with the radiologist worklist/queue and will highlight those CT scans that should be read by the radiologist first, will notify the radiologist to review it by a certain time, or will assign the chest x-ray to a different radiologist (e.g., a general radiologist or radiology sub-specialist). These exam processing algorithms are subject to a certain number of false positive results, which can counteract the benefits the algorithms provide.

A system and method to review a radiology exam is described herein. The system is programmed to analyze the radiology exam by image processing, such as with artificial intelligence, and then, based on the analysis, re-order a review queue, notify a reviewer to review the radiology exam, assign the radiology exam to a specific reviewer, the like, or combinations or multiples thereof. The analysis and subsequent re-order, notification, or assignment can be controlled by review settings. The review settings can also turn the image processing or artificial intelligence on or off.

Algorithmically selecting exams, such as those in a review queue, to send to the exam processing algorithm can reduce the number of exams that need to be processed by the exam processing algorithm, optimize workflow, reduce the number of false positives a radiologist needs to investigate and eliminate from consideration, the like, or combinations or multiples thereof. This can reduce practice costs, such as the cost of initiating the exam processing algorithm, thereby saving time and money. This can also reduce delays in reviewing radiology exams by re-ordering the queue, such that radiology exams from patients having been subject to greater or more life-threatening trauma can be prioritized. The delays can also be reduced by notifying a reviewer that the radiology exam should examined by or within a set time period. The delays can be further reduced by assigning or re-assigning a radiology exam to a different reviewer, including a sub-specialist, whether because of a queue backlog of a first reviewer or because the new reviewer is better suited to review the indicated trauma.

The system includes a radiology server and a communication server. The radiology server is programmed to analyze the radiology exam, control settings, and re-order a review queue, generate a notification, or assign the radiology exam to a specific reviewer. The communication server is programmed to transmit the notification to the reviewer by one or more communication methods or protocols.

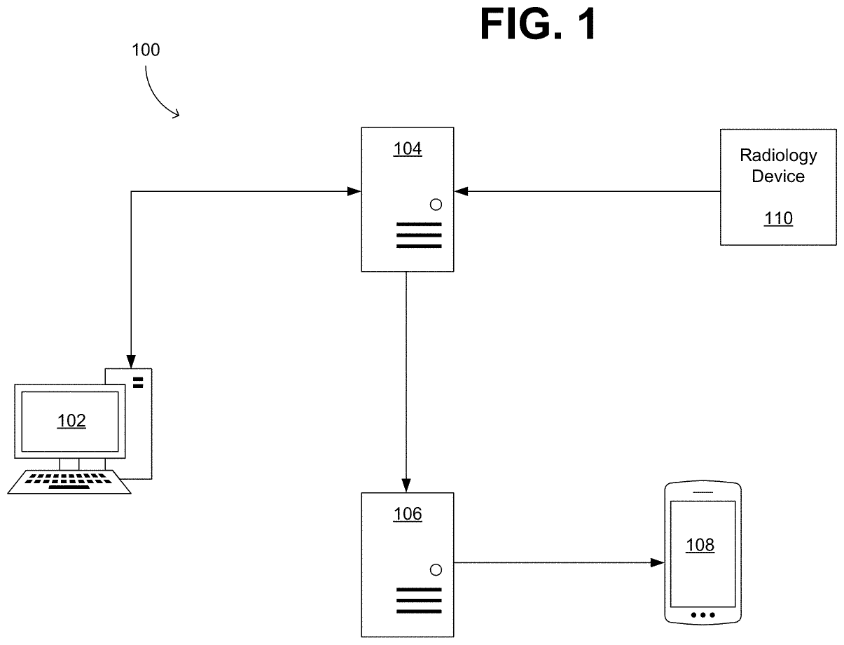

FIG. 1 shows a system 100 for reviewing a radiology exam. A radiology exam includes one or more images or videos of a patient or subject obtained by a scan or imaging exam (e.g., x-rays, magnetic resonance imaging (MM), computed tomography (CT), positron emission tomography (PET) scan, ultrasound, or the like).

The system 100 includes a radiology server 104 and a radiology device 110. The system 100 can also include a communication server 106, a review device 102, and a reviewer user equipment 108. The review device 102 is a device or system for reviewing the radiology exam (e.g., a phone, a smartphone, a tablet, a personal data assistant, a pager, a laptop, a computer, or the like). The reviewer user equipment 108 is a device owned by or in possession of the reviewer capable of receiving a communication (e.g., a phone, a smartphone, a tablet, a personal data assistant, a pager, a laptop, a computer, or the like). Though the review device 102 and the reviewer user equipment 108 can be the same device, the review device 102 is typically on-site (i.e., at the hospital in the lab, in a doctor's office, in a radiologist's office, in an imaging office, or the like) and the reviewer user equipment 108 is typically a personal device of the reviewer.

The radiology server 104 can re-order a review queue, generate a notification, assign the radiology exam to a specific reviewer (e.g., a general radiologist, a radiology sub-specialist, or the like), the like, or combinations or multiples thereof, based on an analysis of a radiology exam and one or more settings or parameters of a review system or device.

The radiology server 104 includes one or more network protocols to communicate with a review device 102, a radiology device 110 (e.g., a system, device, or machine used to perform x-rays, an MM, a CT scan, a PET scan, an ultrasound, or the like), and the communication server 106. The radiology server 104 also includes one or more network protocols to transmit data between the radiology server 104 and the review device 102, radiology device 110, and the communication server 106. For example, the radiology server 104 can include a file transfer protocol (FTP), such as DICOM, to transfer files between the radiology server 104 and a device, computing device, or another server, such as via a TCP/IP connection. A transmission control protocol (TCP) allows for communication over a network, such as by dividing any message into packets which are sent to the destination from the source to be reassembled. An internet protocol (IP) is an addressing protocol, which is mostly used with TCP. The IP addresses of the packets, such as those formed by the TCP, to help route them through the network, such as via one or more nodes, until the packet or packets reach the destination.

The radiology server 104 can also include one or more network protocols for transferring hypertext between two or more systems. These protocols can include hypertext transfer protocol (HTTP) and hypertext transfer protocol secure (HTTPS).

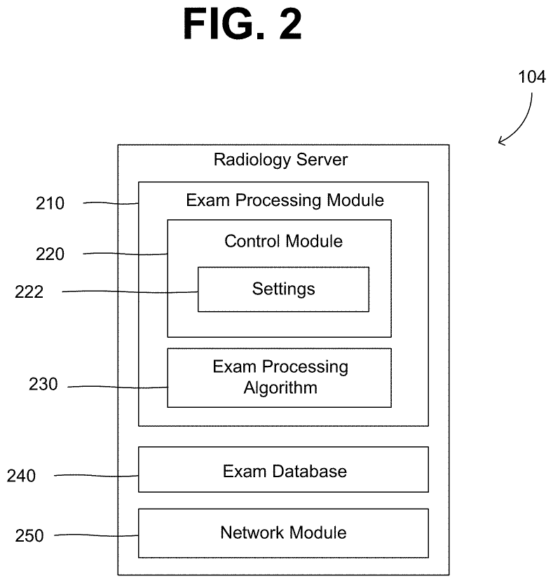

FIG. 2 shows the radiology server 104. The radiology server 104 includes an exam processing module 210 to analyze the radiology exams acquired by and received from the radiology device 110. The exam processing module 210 includes a control module 220 and an exam processing algorithm 230.

The control module 220 includes settings 222 to configure the actions of the system 100 or components of the system (e.g., the review device 102, the communication server 106, or the radiology server 104). For example, the settings 220 can set conditions by which the radiology exam should or should not be processed by the exam processing database 230. As another example, the settings 220 can set conditions by which a radiology exam review queue should be re-ordered, a notification should be generated to notify a reviewer that a radiology exam needs review (e.g., review should be performed by a certain time or within a certain amount of time), the radiology exam should be assigned to the specific reviewer, or the like.

Based on the settings 222 or the settings 222 and the radiology exam analysis, the control module 220 can generate instructions for another system component or another component of the radiology server 104. For example, the control module 220 can generate a notification to be transmitted to the reviewer, such as to the reviewer device 102 or to the reviewer user equipment 108 via the communication server 106 to review a radiology exam as soon as possible, within a certain amount of time (e.g., minutes, hours, or days), by a certain time (e.g., time of the day or a date), or the like. As another example, the control module 220 can generate and transmit an instruction to the review device 102 to re-order a list of radiology exams (i.e., move a current radiology exam to the top of list, change locations of other radiology exams, or the like). Or, the control module 220 can generate an instruction for the exam database 240 to re-order a list of radiology exams (i.e., move a current radiology exam to the top of list, change locations of other radiology exams, or the like). As yet another example, the control module 220 can generate an instruction to the network module 250 to transmit the radiology exam to a specific reviewer.

The exam processing algorithm 230 is an algorithm to perform image or video processing, such as with artificial intelligence. The algorithm can perform computer-aided simple triage (CAST; i.e., perform initial interpretation and triage of the radiology exam and classify the radiology exam into a category—for example, positive or negative), computer-aided detection (CADe; i.e., analyze the radiology exam and highlight or flag an area of the radiology exam for further review), or computer-aided diagnostic (CADx; i.e., analyze the radiology exam and identify the nature of an illness or problem). For example, companies such as AIDoc and Zebra Medical have artificial intelligence for radiology exams.

The radiology server 104 also includes an exam database 240 that stores radiology exams and any associated information, including patient name or identifier, results of the exam processing algorithm 230, the like, or combinations or multiples thereof. The data and information can be stored in any appropriate format, standard, or table. For example, the data and information can be stored in comma separated value tables, fielded text, data interchange format, HTML sourced data, JQuery, Bootstrap, or the like.

The radiology server 104 also includes a communication module 250 which is programmed to communicate, via one or more protocols (including one or more network protocols), with another device, such as the review device 102, the radiology device 110, and the communication server 106, through a physical connection, such as a local area network (LAN), Universal Serial Bus (USB), or the like, or a wireless connection, such as Bluetooth®, WiFi, wireless networks, or the like.

Returning to FIG. 1, the communication server 106 is programmed to transmit the notification from the radiology server 104 to the reviewer user equipment 108 by one or more communication methods or protocols. The notification can be transmitted by the communication server 106 by e-mail, text (including real time text), audio (including with touch tones), or any appropriate method or protocol by which a notification can transmitted and received.

The communication server 106 includes one or more network protocols to communicate with the radiology server 104 and the review user equipment 108. The communication server 106 also includes one or more network protocols to transmit data between the communication server 106 and the radiology server 104 or the review user equipment 108. For example, the communication server 106 can include TCP, IP, or both.

The communication server 106 can also include one or more network protocols for transferring hypertext between two or more systems, such as HTTP or HTTPS.

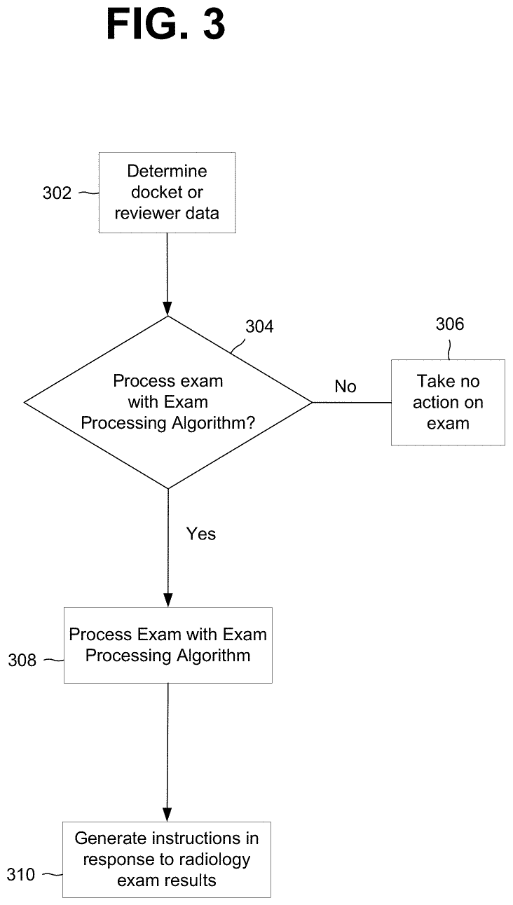

FIG. 3 shows a method for reviewing a radiology exam. In some embodiments, the system and methods process and queue radiology exams. For example, the radiology exams are processed after entering the queue. This processing occurs when the system deems such processing necessary based on evaluation of one or more rules or decision processes. The system implements an algorithm or decision process (e.g., the exam processing module 210) that uses data regarding the current state of exam “clearance rate” or “turnaround time”, as well as radiologist scheduling information, in combination with a series of programmable rules, to determine if processing is necessary (and when to implement that processing).

At 302, data associated with a review docket, with a reviewer, or with the review docket and the reviewer is acquired and analysed. This data includes clearance rate of radiology exams by an individual reviewer or a team of reviewers (i.e., rate by which radiology exams are reviewed and therefore removed from the queue of radiology exams to be reviewed), the length of the radiology exam queue (i.e., the number of radiology exams to be reviewed), the reviewer to whom the radiology exam is assigned, the like, or combinations or multiples thereof.

At 304, a decision is made as whether or not a radiology exam is process with the exam processing algorithm. To make this determination, the data associated with the reviewer docket, the assigned reviewer, the radiology exam, the queue settings (i.e., how to process the queue), the like, or combinations of multiples thereof, is analysed.

The queue settings include a series of programmable rules to determine which exams to send to the exam processing algorithm, and when it should send those exams. As an example, a rule could be based upon the size of the queue of exams waiting to be read (i.e., reviewed and evaluated). In this example, if a queue of unread chest X-Rays exceeded a specified threshold number, then the system automatically sends those exams to the exam processing algorithm. This ensures that exams of patients with critical conditions get read first. In this example, if the queue remained under the threshold number, then no exams would be sent to the exam processing algorithm because there is no clinical need to accelerate the reading of those exams. That is, the radiology practice is caught up, so the radiology exams will be read in a timely manner.

Further examples of queue settings, such as in the form of logic or decision rules, that can include a turnaround time for certain exams is currently exceeding a threshold, a reason for the exam is because of a condition such as “trauma,” patient information, such as patient age, a procedure code or other metadata such as site or location, queue of exams exceeds a threshold, exam is assigned (or is expected to be read by) a specific radiologist, the like, or combinations or multiples thereof.

As a triage example, a busy practice may be clearing chest X-Rays at the rate of 20/hour; further, assume there are 20 in the queue to be read. In addition, assume that historical information allows one to predict that another 20 chest X-Rays will arrive in the next hour. Based upon the radiologists' work schedule, one might predict that staffed radiologists can read 30 exams in the next hour. Based on the assumptions mentioned, this would result in a deficit of 10 exams which will not be read in the next hour. Historically, one may expect to find that 1 out of these 10 “unread” exams to have a critical condition. In this case, the algorithm can send the 11 exams at the bottom of the queue to be processed to uncover if there is a possible critical condition. If the algorithm detects 1 exam with a critical condition, that exam can be moved to the top of the list to be read, and the 10 exams that have been “cleared” can stay at the bottom of the list.

As a CADe or CADx example, the radiology practice can decide to not use the exam processing algorithm when a sub-specialist is reading an exam within their sub-specialty (such as a neuroradiologist reading a head CT), but to use the exam processing algorithm when the exam will be read by someone outside their subspecialty or by a general radiologist.

At 306, if it is determined that the radiology exam does not need to be processed by the exam processing algorithm, then no action is taken on the radiology exam. The radiology exam remains at its current location within the queue.

At 308, if it is determined that the radiology exam does need to be processed by the exam processing algorithm, the radiology exam is transmitted to or retrieved by the exam processing algorithm and the exam processing algorithm analysed the radiology exam.

After the radiology exam has been analysed, an action can be taken in response to the results of the analysis. At 310, the system can generate instructions based on the results of the analysis of the radiology exam by the exam processing algorithm. The instructions can also be based on one or more of the queue settings. The instructions can be generated in the form of a signal. The instructions can effect a change on a system component, such as by re-ordering the exam queue on the radiology server 104), by transmitting the radiology exam to another device or server, by transmitting a notification to the server (e.g., cause review device 102 or the communication server 106 to transmit the notification), by moving the radiology exam up in the queue, the like, or combinations or multiples thereof. The radiology exam can be moved up in the queue (i.e., moved to the top or to a higher priority spot within the queue than the place from which the radiology originated), the radiologist can be notified to review the radiology exam within a certain time frame, the radiology exam can be assigned or re-assigned to a more appropriate reviewer, the like, or combinations or multiples thereof.

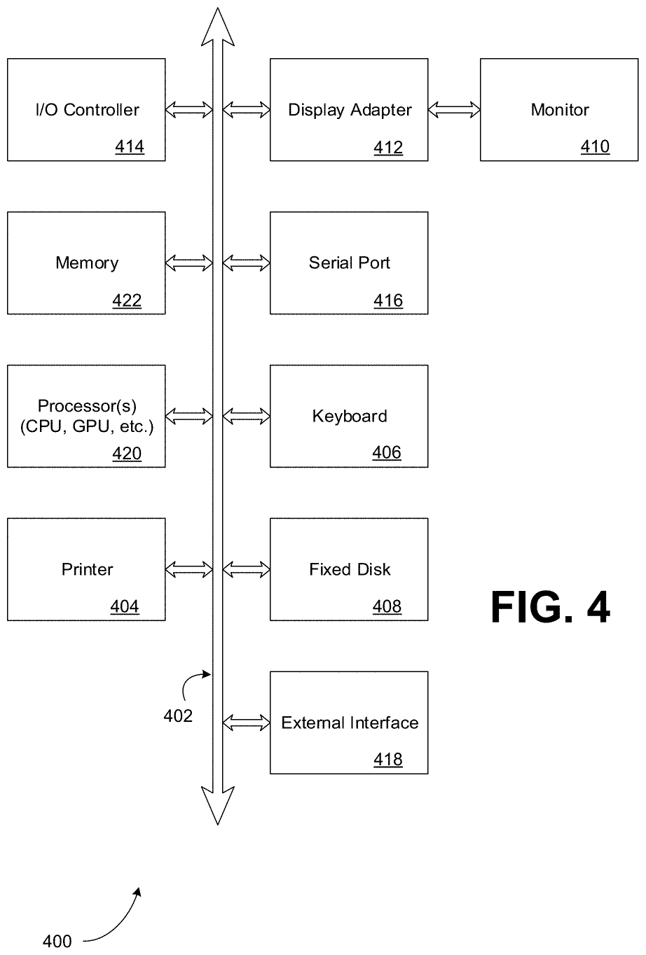

As an example, FIG. 4 is a diagram illustrating elements or components that can be present in a computer device or system 400 configured to implement a method, process, function, or operation in accordance with an example of the invention. The subsystems shown in FIG. 4 are interconnected via a system bus 402. Additional subsystems include a printer 404, a keyboard 406, a fixed disk 408, and a monitor 410, which is coupled to a display adapter 412. Peripherals and input/output (I/O) devices, which couple to an I/O controller 414, can be connected to the computer system by any number of means known in the art, such as a serial port 416. For example, the serial port 416 or an external interface 418 can be utilized to connect the computer device 400 to further devices or systems not shown in FIG. 4 including a wide area network such as the Internet, a mouse input device, a document scanner, the like, or combinations or multiples thereof. The interconnection via the system bus 402 allows one or more electronic processors 420 to communicate with each subsystem and to control the execution of instructions that can be stored in a system memory 422, the fixed disk 408, or both, as well as the exchange of information between subsystems. The system memory 422, the fixed disk 408, or both can embody a tangible computer-readable medium.

Note that the example computing environments depicted in the figures and described herein are not intended to be limiting examples. Alternatively, or in addition, computing environments in which an embodiment of the invention may be implemented include any suitable system that permits users to provide data to, and access, process, and utilize data stored in a data storage element (e.g., a database) that can be accessed remotely over a network. Further example environments in which an embodiment of the invention may be implemented include devices (including mobile devices), software applications, systems, apparatuses, networks, or other configurable components that may be used by multiple users for data entry, data processing, application execution, data review, etc. and which have user interfaces or user interface components that can be configured to present an interface to a user. Although further examples may reference the example computing environment depicted in the Figures, it will be apparent to one of skill in the art that the examples may be adapted for alternate computing devices, systems, apparatuses, processes, and environments.

Among other things, the present invention can be embodied in whole or in part as a system, as one or more methods, or as one or more devices. Examples of the invention can take the form of a hardware-implemented example, a software implemented example, or an example combining software and hardware aspects. For example, in some examples, one or more of the operations, functions, processes, or methods described herein can be implemented by one or more suitable processing elements (such as a processor, microprocessor, CPU, GPU, controller, etc.) that is part of a client device, server, network element, or other form of computing or data processing device/platform. The processing element or elements are programmed with a set of executable instructions (e.g., software instructions), where the instructions can be stored in a suitable data storage element. In some examples, one or more of the operations, functions, processes, or methods described herein can be implemented by a specialized form of hardware, such as a programmable gate array (PGA or FPGA), application specific integrated circuit (ASIC), or the like. Note that an example of the inventive methods can be implemented in the form of an application, a sub-routine that is part of a larger application, a “plug-in,” an extension to the functionality of a data processing system or platform, or other suitable form.

It should be understood that the present invention as described above can be implemented in the form of control logic using computer software in a modular or integrated manner. Based on the disclosure and teachings provided herein, a person of ordinary skill in the art will know and appreciate other ways or methods to implement the present invention using hardware and a combination of hardware and software.

Different arrangements of the components depicted in the drawings or described above, as well as components and steps not shown or described are possible. Similarly, some features and sub-combinations are useful and may be employed without reference to other features and sub-combinations. Embodiments of the invention have been described for illustrative and not restrictive purposes, and alternative embodiments will become apparent to readers of this patent. Accordingly, the present invention is not limited to the embodiments described above or depicted in the drawings, and various embodiments and modifications can be made without departing from the scope of the claims below.

Any of the software components, processes or functions described in this application may be implemented as software code to be executed by a processor using any suitable computer language such as, for example, Python, Java, JavaScript, C++ or Perl using, for example, conventional or object-oriented techniques. The software code may be stored as a series of instructions, or commands in (or on) a non-transitory computer-readable medium, such as a random-access memory (RAM), a read only memory (ROM), a magnetic medium such as a hard-drive or a floppy disk, or an optical medium such as a CD-ROM. In this context, a non-transitory computer-readable medium is almost any medium suitable for the storage of data or an instruction set aside from a transitory waveform. Any such computer readable medium may reside on or within a single computational apparatus and may be present on or within different computational apparatuses within a system or network.

According to one example implementation, the term processing element or processor, as used herein, may be a central processing unit (CPU), or conceptualized as a CPU (such as a virtual machine). In this example implementation, the CPU or a device in which the CPU is incorporated may be coupled, connected, or in communication with one or more peripheral devices, such as display. In another example implementation, the processing element or processor may be incorporated into a mobile computing device, such as a smartphone or tablet computer.

The non-transitory computer-readable storage medium referred to herein may include a number of physical drive units, such as a redundant array of independent disks (RAID), a floppy disk drive, a flash memory, a USB flash drive, an external hard disk drive, thumb drive, pen drive, key drive, a High-Density Digital Versatile Disc (HD-DV D) optical disc drive, an internal hard disk drive, a Blu-Ray optical disc drive, or a Holographic Digital Data Storage (HDDS) optical disc drive, synchronous dynamic random access memory (SDRAM), or similar devices or other forms of memories based on similar technologies. Such computer-readable storage media allow the processing element or processor to access computer-executable process steps, application programs and the like, stored on removable and non-removable memory media, to off-load data from a device or to upload data to a device. As mentioned, with regards to the embodiments described herein, a non-transitory computer-readable medium may include almost any structure, technology or method apart from a transitory waveform or similar medium.

Certain implementations of the disclosed technology are described herein with reference to block diagrams of systems, to flowcharts or flow diagrams of functions, operations, processes, or methods, or the like. It will be understood that one or more blocks of the block diagrams, or one or more stages or steps of the flowcharts or flow diagrams, and combinations of blocks in the block diagrams and stages or steps of the flowcharts or flow diagrams, respectively, can be implemented by computer-executable program instructions. Note that in some embodiments, one or more of the blocks, or stages or steps may not necessarily need to be performed in the order presented or may not necessarily need to be performed at all.

These computer-executable program instructions may be loaded onto a general-purpose computer, a special purpose computer, a processor, or other programmable data processing apparatus to produce a specific example of a machine, such that the instructions that are executed by the computer, processor, or other programmable data processing apparatus create means for implementing one or more of the functions, operations, processes, or methods described herein. These computer program instructions may also be stored in a computer-readable memory that can direct a computer or other programmable data processing apparatus to function in a specific manner, such that the instructions stored in the computer-readable memory produce an article of manufacture including instruction means that implement one or more of the functions, operations, processes, or methods described herein.

Though certain elements, aspects, components or the like are described in relation to one embodiment or example, such as an example system, those elements, aspects, components or the like can be including with any other systems, such as when it desirous or advantageous to do so.

The foregoing description, for purposes of explanation, used specific nomenclature to provide a thorough understanding of the disclosure. However, it will be apparent to one skilled in the art that the specific details are not required in order to practice the systems and methods described herein. The foregoing descriptions of specific embodiments are presented by way of examples for purposes of illustration and description. They are not intended to be exhaustive of or to limit this disclosure to the precise forms described. Many modifications and variations are possible in view of the above teachings. The embodiments are shown and described in order to best explain the principles of this disclosure and practical applications, to thereby enable others skilled in the art to best utilize this disclosure and various embodiments with various modifications as are suited to the particular use contemplated. It is intended that the scope of this disclosure be defined by the following claims and their equivalents.

What is claimed is:

1. A system for processing a radiology exam, the system comprising:
a processor programmed to:
determine the radiology exam to be analyzed based on a first parameter,
analyze the radiology exam with an exam processing algorithm,
generate an instruction based on the analysis of the radiology exam; and
an output programmed to output the instruction to effect a change on a system component associated with the radiology exam.

2. The system of claim 1, wherein the first parameter is a characteristic of the radiology exam, a radiology review turnaround time exceeding a threshold, a reason for the radiology exam, a procedure code or other metadata, a queue length of radiology exams exceeding a threshold, reviewer assignment, a clearance rate of radiology exam queue, or combinations or multiples thereof.

3. The system of claim 1, wherein the exam processing algorithm is computer-aided simple triage, computer-aided detection, or computer-aided diagnostic.

4. The system of claim 1, wherein the instruction causes the radiology exam to be re-ordered within a radiology exam queue.

5. The system of claim 4, wherein the radiology exam is moved up in the queue.

6. The system of claim 5, wherein the radiology exam is moved to the top of the queue.

7. The system of claim 1, wherein the instruction causes the radiology exam to be assigned to a specific reviewer.

8. The system of claim 1, wherein the instruction causes the radiology exam to be re-assigned to a different reviewer.

9. The system of claim 1, wherein the instruction causes a notification to be transmitted to a device of a reviewer.

10. The system of claim 9, wherein the notification directs the reviewer to review the radiology exam within a given amount of time or by a given time.

11. The system of claim 1, wherein the radiology exam is one of a plurality of radiology exams within a radiology exam queue.

12. The system of claim 11, wherein another radiology exam of the plurality of radiology exams is not analyzed by the exam processing algorithm.

13. The system of claim 11, wherein another radiology exam of the plurality of radiology exams is analyzed by the exam processing algorithm.

14. The system of claim 1, wherein the processor is further programmed to turn the exam processing algorithm on or off based on a second parameter.

15. The system of claim 14, wherein the second parameter is a radiology review turnaround time, a queue length of radiology exams, clearance rate of a radiology exam queue, or combinations or multiples thereof.

16. A method for processing a radiology exam, the system comprising:
determining the radiology exam to be analysed based on a first parameter,
analyzing the radiology exam with an exam processing algorithm,
generating an instruction based on the analysis of the radiology exam; and
outputting the instruction.

17. The method of claim 16, wherein the first parameter is a characteristic of the radiology exam, a radiology review turnaround time exceeding a threshold, a reason for the radiology exam, a procedure code or other metadata, a queue length of radiology exams exceeding a threshold, reviewer assignment, a clearance rate of radiology exam queue, or combinations or multiples thereof.

18. The method of claim 16, wherein the instruction causes the radiology exam to be re-ordered within a radiology exam queue.

19. The method of claim 16, wherein the instruction causes the radiology exam to be assigned to a specific reviewer or to be re-assigned to a different reviewer.

20. The method of claim 16, wherein the instruction causes a notification to be transmitted to a device of a reviewer to direct the reviewer to review the radiology exam within a given amount of time or by a given time.
